(19) United States
(12) Patent Application Publication
(10) Pub. No.: US 2021/0277434 A1
(43) Pub. Date: Sep. 9, 2021

(54) GLUCOAMYLASES AND METHODS OF USE, THEREOF

(71) Applicant: DANISCO US INC, Palo Alto, CA (US)

(72) Inventors: Zhongmei TANG, Shanghai (CN); Qihui WU, Shanghai (CN); Xingxiang XI, Shanghai (CN); Zhenghong ZHANG, Shanghai (CN)

(21) Appl. No.: 17/257,641

(22) PCT Filed: Jul. 2, 2019
(86) PCT No.: PCT/US2019/040331

§ 371 Date: Jan. 4, 2021

(30) Foreign Application Priority Data
Jul. 4, 2018 (CN) PCT/CN2018/094473

(51) Int. Cl. C12P 19/14; C12P 19/02

(52) U.S. Cl. CPC C12P 19/14; C12Y 302/01003; C12P 19/02

(57) ABSTRACT

Described are methods of saccharifying starch-containing materials using a glucoamylase, the methods of producing fermentation products and the fermentation products produced by the method thereof.

### FIELD OF THE INVENTION

The present disclosure relates to methods of saccharifying starch-containing materials using at least one glucoamylase. Moreover, the disclosure also relates to methods of producing fermentation products as well as the fermentation products produced by the method thereof.

### BACKGROUND

Glucoamylases (GAs, EC 3.2.1.3) are multidomain exoglucohydrolases that consecutively hydrolyzes α-1,4 glycosidic bonds from the nonreducing ends of starch, resulting in the production of glucose. Glucoamylases are produced by several filamentous fungi and yeast.

The major application of glucoamylase is the saccharification of partially processed starch/dextrin to glucose, which is an essential substrate for numerous fermentation processes. The glucose may then be converted directly or indirectly into a fermentation product using a fermenting organism. Examples of commercial fermentation products include alcohols (e.g., ethanol, methanol, butanol, 1,3-propanediol); organic acids (e.g., citric acid, acetic acid, itaconic acid, lactic acid, gluconic acid, gluconate, lactic acid, succinic acid, 2,5-diketo-D-gluconic acid); ketones (e.g., acetone); amino acids (e.g., glutamic acid); gases (e.g., H₂ and CO₂), and more complex compounds.

The end product may also be syrup. For instance, the end product may be glucose, but may also be converted, e.g., by glucose isomerase to fructose or a mixture composed almost equally of glucose and fructose. This mixture, or a mixture further enriched with fructose, is the most commonly used high fructose corn syrup (HFCS) commercialized throughout the world.

Glucoamylase for commercial purposes has traditionally been produced employing filamentous fungi, although a diverse group of microorganisms is reported to produce glucoamylase, since they secrete large quantities of the enzyme extracellularly. However, the commercially used fungal glucoamylases have certain limitations such as moderate thermostability, acidic pH instability, slow catalytic activity that increase the process cost.

Accordingly, there is a need to search for new glucoamylases to improve the thermostability, pH stability or efficiency of saccharification to provide a high yield in glucose, fermentation products, such as biochemicals, ethanol production, including one-step ethanol fermentation processes from un-gelatinized raw (or uncooked) starch.

### SUMMARY

The present disclosure relates to methods of saccharifying starch-containing materials using at least one Penicillum or Symbiotaphrina glucoamylase. Moreover, the disclosure also relates to methods of producing fermentation products as well as the fermentation products produced by the method thereof. Aspects and embodiments of the compositions and methods are described in the following, independently-numbered, paragraphs.

1. In one aspect, a method for saccharification of a starch substrate is provided, comprising contacting the substrate with a glucoamylase having at least two, at least three, or at least four times more activity on an α-1,6 bond-containing substrate compared to the glucoamylase from Aspergillus niger under equivalent conditions, wherein saccharifying with the glucoamylase produces a glucose syrup having a higher level of glucose compared to saccharifying the same starch substrate with the glucoamylase from Aspergillus niger under equivalent conditions.

2. In another aspect, a method for increasing the amount of glucose in a syrup produced by saccharifying a starch substrate is provided, comprising contacting the substrate with a glucoamylase having at least two, at least three, or at least four times more activity on an α-1,6 bond-containing substrate compared to the glucoamylase from Aspergillus niger under equivalent conditions, wherein the saccharifying with the glucoamylase produces a glucose syrup having a higher level of glucose compared to saccharifying the same starch substrate with the glucoamylase from Aspergillus niger under equivalent conditions.

3. In some embodiments of the method of paragraph 1 or 2, the α-1,6 bond-containing substrate is amylopectin, panose or isomaltose.

4. In some embodiments of the method of any of the preceding paragraphs, the glucoamylase has at least 20% more activity at pH 4.5 on soluble starch substrate compared to the glucoamylase from Aspergillus niger under equivalent conditions.

5. In some embodiments of the method of any of the preceding paragraphs, the glucose syrup comprises at least 4%, at least 10%, or at least 25% more glucose compared to a syrup produced by saccharifying with the glucoamylase from Aspergillus niger at a temperature between 60 and 69° C.

6. In some embodiments of the method of any of the preceding paragraphs, the glucose syrup comprises at least a 4% reduction, at least a 10% reduction, or at least a 20% reduction in DP3+ compared to a glucose syrup prepared by contacting the same starch substrate with the glucoamylase from Aspergillus niger under equivalent conditions.

7. In some embodiments of the method of any of the preceding paragraphs, the glucose syrup comprises at least 90% glucose, at least 91% glucose, at least 92% glucose, at least 93% glucose, at least 94% glucose, at least 95% glucose, at least 96% glucose, at least 97% glucose, at least 98% glucose or at least 99% glucose.

8. In some embodiments of the method of any of the preceding paragraphs, saccharifying the starch substrate is performed at a temperature above 60° C., above 65° C., above 70° C., above 75° C., or above 80° C.

9. In some embodiments of the method of any of the preceding paragraphs, saccharifying the starch substrate is performed at a pH below 4.5, below 4.0, or below 3.5.

10. In some embodiments of the method of any of the preceding paragraphs, performed within a simultaneous saccharification and fermentation process.

11. In some embodiments of the method of any of the preceding paragraphs, the glucoamylase has at least 50% residual activity at 80° C. after 10 minutes at pH 5.0.

12. In some embodiments of the method of any of the preceding paragraphs, the glucoamylase has at least 20% more activity at pH 3 compared to the glucoamylase from Aspergillus niger under equivalent conditions.

13. In some embodiments of the method of any of the preceding paragraphs, the glucoamylase is from Penicillium glabrum, Symbiotaphrina kochii, Penicillium brasilianum or a variant, thereof.

14. In some embodiments of the method of any of the preceding paragraphs, the glucoamylase is selected from the groups consisting of:
a) a polypeptide having the amino acid sequence of SEQ ID NO: 2, SEQ ID NO: 4, or SEQ ID NO: 6;
b) a polypeptide having at least 80% identity to the amino acid sequence of SEQ ID NO: 2, SEQ ID NO: 4, or SEQ ID NO: 6; or
c) a polypeptide having at least 80% identity to a catalytic domain of SEQ ID NO: 2, SEQ ID NO: 4, or SEQ ID NO: 6.

15. In another aspect, a recombinant construct comprising a nucleotide sequence encoding a glucoamylase is provided, said coding nucleotide sequence is operably linked to at least one regulatory sequence functional in a production host and is selected from the group consisting of the nucleotide sequence set forth in SEQ ID NO: 1, SEQ ID NO: 3, or SEQ ID NO: 5 or a nucleotide sequence with at least 80% sequence identity thereto, wherein said regulatory sequence is heterologous to the coding nucleotide sequence, or said regulatory sequence and coding sequence are not arranged as found together in nature.

These and other aspects and embodiments of present modified cells and methods will be apparent from the description, including any accompanying Drawings/Figures.

### BRIEF DESCRIPTION OF THE SEQUENCES

The following sequences comply with 37 C.F.R. §§ 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the European Patent Convention (EPC) and the Patent Cooperation Treaty (PCT) Rules 5.2 and 49.5(a-bis), and Section 208 and Annex C of the Administrative Instructions. The symbols and format used for nucleotide and amino acid sequence data comply with the rules set forth in 37 C.F.R. § 1.822.

SEQ ID NO: 1 is nucleotide sequence of the Penicillum glabrum PglGA1 synthetic gene.

SEQ ID NO: 2 is amino acid sequence of the Penicillum glabrum PglGA1 mature protein.

SEQ ID NO: 3 is nucleotide sequence of the Symbiotaphrina kochii SkoGA1 synthetic gene.

SEQ ID NO: 4 is amino acid sequence of the Symbiotaphrina kochii SkoGA1 mature protein.

SEQ ID NO: 5 is nucleotide sequence of the Penicillium brasilianum PhrGA5 synthetic gene.

SEQ ID NO: 6 is amino acid sequence of the Penicillium brasilianum PbrGA5 mature protein.

SEQ ID NO: 7 is amino acid sequence of the wild type glucoamylase from Aspergillus niger, and the NCBI accession number is XP_001390530.1.

SEQ ID NO: 8 is amino acid sequence of the wild type glucoamylase from Trichoderma reesei, and the PDB accession number is 2VN4_A.

SEQ ID NO: 9 is amino acid sequence of the wild type alpha-amylase from Aspergillus kawachii, and the NCBI accession number is BAA22993.1.

### DETAILED DESCRIPTION

### Definitions and Abbreviations

All patents, patent applications, and publications cited are incorporated herein by reference in their entirety. In this disclosure, a number of terms and abbreviations are used. The following definitions apply unless specifically stated otherwise.

The term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”. As used herein in connection with a numerical value, the term “about” refers to a range of +/−0.5 of the numerical value, unless the term is otherwise specifically defined in context. For instance, the phrase a “pH value of about 6” refers to pH values of from 5.5 to 6.5, unless the pH value is specifically defined otherwise.

Unless otherwise defined, all technical and scientific terms used have their ordinary meaning in the relevant scientific field. Singleton, et al., Dictionary of Microbiology and Molecular Biology, 2d Ed., John Wiley and Sons, New York (1994), and Hale & Markham, Harper Collins Dictionary of Biology, Harper Perennial, N.Y. (1991) provide the ordinary meaning of many of the terms describing the invention.

The term “glucoamylase (1,4-alpha-D-glucan glucohydrolase, EC 3.2.1.3) activity” is defined herein as an enzyme activity, which catalyzes the release of D-glucose from the non-reducing ends of starch or related oligo- and poly-saccharide molecules. The majority of glucoamylases are multidomain enzymes consisting of a catalytic domain connected to a starch binding domain by an O-glycosylated linker region of varying lengths. The crystal structures of multiple glucoamylases have been determined and described (see J. Lee and M. Paetzel 2011. Acta Cryst. 67:188-92 and J. Sauer et al 2000. Biochem. Et Biophys. Acta 1542:275-93.

The terms “starch binding domain (SBD) or carbohydrate binding module (CBM)” are used interchangeably herein. SBDs can be divided into nine CBM families. As a source of energy, starch is degraded by a large number of various amylolytic enzymes. However, only about 10% of them are capable of binding and degrading raw starch. These enzymes usually possess a distinct sequence-structural module called the starch-binding domain that mediates attachment to starch granules. SBD refers to an amino acid sequence that binds preferentially to a starch (polysaccharide) substrate or a maltosaccharide, alpha-, beta and gamma-cyclodextrin and the like. They are usually motifs of approximately 100 amino acid residues found in about 10% of microbial amylolytic enzymes.

The term “catalytic domain (CD)” refers to a structural region of a polypeptide which contains the active site for substrate hydrolysis.

The term “glycoside hydrolase” is used interchangeably with “glycosidases” and “glycosyl hydrolases”. Glycoside hydrolases assist in the hydrolysis of glycosidic bonds in complex sugars (polysaccharides). Glycoside hydrolases can also be classified as exo- or endo-acting, dependent upon whether they act at the (usually non-reducing) end or in the middle, respectively, of an oligo/polysaccharide chain. Glycoside hydrolases may also be classified by sequence or structure based methods.

The term “α-1,6 bond-containing substrate” refers to oligosaccharides or polysaccharides that contain at least one α-1,6 bond, and can be hydrolyzed by a glycosyl hydrolase. Examples of α-1,6 bond-containing substrates include, but are not limited to: isomaltose, panose, isomaltotriose, and pullulan.

The term “granular starch” refers to raw (uncooked) starch, e.g., granular starch that has not been subject to gelatinization.

The terms “granular starch hydrolyzing (GSH) enzyme” and “granular starch hydrolyzing (GSH) activity” are used interchangeably herein and refer to enzymes, which have the ability to hydrolyze starch in granular form under digestive tract relevant conditions comparable to the conditions found in the digestive tract of animals and, in particular, ruminants.

The term “isolated” means a substance in a form or environment that does not occur in nature. Non-limiting examples of isolated substances include (1) any non-naturally occurring substance, (2) any substance including, but not limited to, any host cell, enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated. The terms “isolated nucleic acid molecule”, “isolated polynucleotide”, and “isolated nucleic acid fragment” will be used interchangeably and refer to a polymer of RNA or DNA that is single- or double-stranded, optionally containing synthetic, non-natural or altered nucleotide bases.

The term “purified” as applied to nucleic acids or polypeptides generally denotes a nucleic acid or polypeptide that is essentially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.”

The terms “peptides”, “proteins” and “polypeptides” are used interchangeably herein and refer to a polymer of amino acids joined together by peptide bonds. A “protein” or “polypeptide” comprises a polymeric sequence of amino acid residues. The single and 3-letter code for amino acids as defined in conformity with the IUPAC-IUB Joint Commission on Biochemical Nomenclature (JCBN) is used throughout this disclosure. It is also understood that a polypeptide may be coded for by more than one nucleotide sequence due to the degeneracy of the genetic code.

The term “mature” form of a protein, polypeptide, or enzyme refers to the functional form of the protein, polypeptide, or enzyme without a signal peptide sequence or a propeptide sequence.

The term “precursor” form of a protein or peptide refers to a form of the protein having a prosequence operably linked to the amino or carbonyl terminus of the protein. The precursor may also have a “signal” sequence operably linked to the amino terminus of the prosequence.

The term “percent identity” is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the number of matching nucleotides or amino acids between strings of such sequences. “Identity” and “similarity” can be readily calculated by known methods, including but not limited to those described in: Computational Molecular Biology (Lesk, A. M., ed.) Oxford University Press, N Y (1988); Biocomputing: Informatics and Genome Projects (Smith. D. W., ed.) Academic Press, N Y (1993); Computer Analysis of Sequence Data, Part I(Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Methods to determine identity and similarity are codified in publicly available computer programs. Percent identity may be determined using standard techniques known in the art. Useful algorithms include the BLAST algorithms (See, Altschul et al., J Mol Biol, 215:403-410, 1990; and Karlin and Altschul, Proc Natl Acad Sci USA, 90:5873-5787, 1993). The BLAST program uses several search parameters, most of which are set to the default values. The NCBI BLAST algorithm finds the most relevant sequences in terms of biological similarity but is not recommended for query sequences of less than 20 residues (Altschul et al., Nucleic Acids Res, 25:3389-3402, 1997; and Schaffer et al., Nucleic Acids Res, 29:2994-3005, 2001). Exemplary default BLAST parameters for a nucleic acid sequence searches include: Neighboring words threshold=11; E-value cutoff=10; Scoring Matrix=NUC.3.1 (match=1, mismatch=−3); Gap Opening=5; and Gap Extension=2. Exemplary default BLAST parameters for amino acid sequence searches include: Word size=3; E-value cutoff=10; Scoring Matrix=BLOSUM62; Gap Opening=11; and Gap extension=1. A percent (%) amino acid sequence identity value is determined by the number of matching identical residues divided by the total number of residues of the “reference” sequence including any gaps created by the program for optimal/maximum alignment. BLAST algorithms refer to the “reference” sequence as the “query” sequence.

As used herein, “homologous proteins” or “homologous enzymes” refers to proteins that have distinct similarity in primary, secondary, and/or tertiary structure. Protein homology can refer to the similarity in linear amino acid sequence when proteins are aligned. Homologous search of protein sequences can be done using BLASTP and PSI-BLAST from NCBI BLAST with threshold (E-value cut-off) at 0.001. (Altschul S F, Madde T L, Shaffer A A, Zhang J. Zhang Z, Miller W, Lipman D J. Gapped BLAST and PSI BLAST a new generation of protein database search programs. Nucleic Acids Res 1997 Set 1; 25(17):3389-402). Using this information, proteins sequences can be grouped, and a phylogenetic tree can also be built using the amino acid sequences. Sequence alignments and percent identity calculations may also be performed using the Megalign program, the AlignX program, the EMBOSS Open Software Suite (EMBL-EBI; Rice et al., Trends in Genetics 16, (6):276-277 (2000)) or similar programs. Multiple alignment of the sequences can also be performed using the CLUSTAL method (such as CLUSTALW) with the default parameters. Suitable parameters for CLUSTALW protein alignments include GAP Existence penalty=15, GAP extension=0.2, matrix=Gonnet (e.g., Gonnet250), protein ENDGAP=−1, protein GAPDIST=4, and KTUPLE=1.

The term “nucleic acid” encompasses DNA. RNA, heteroduplexes, and synthetic molecules capable of encoding a polypeptide. Nucleic acids may be single stranded or double stranded, and may be chemically modified. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

The term “coding sequence” means a nucleotide sequence, which directly specifies the amino acid sequence of its protein product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant nucleotide sequence.

A “synthetic” molecule is produced by in vitro chemical or enzymatic synthesis rather than by an organism.

The terms “recombinant construct,” “expression construct,” “recombinant expression construct” and “expression cassette” are used interchangeably herein. A recombinant construct comprises an artificial combination of nucleic acid fragments, e.g., regulatory and coding sequences that are not all found together in nature. For example, a construct may comprise regulatory sequences and coding sequences that are derived from different sources, or regulatory sequences and coding sequences derived from the same source, but arranged in a manner different than that found in nature. Such a construct may be used by itself or may be used in conjunction with a vector.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

The term “regulatory sequences” is defined herein to include all components necessary for the expression of a polynucleotide encoding a polypeptide of the present invention. Each regulatory sequence may be native or foreign to the nucleotide sequence encoding the polypeptide or native or foreign to each other. Such regulatory sequences include, but are not limited to, a leader, polyadenylation sequence, propeptide sequence, promoter, signal peptide sequence, and transcription terminator. At a minimum, the regulatory sequences include a promoter, and transcriptional and translational stop signals. The regulatory sequences may be provided with linkers for the purpose of introducing specific restriction sites facilitating ligation of the regulatory sequences with the coding region of the nucleotide sequence encoding a polypeptide.

A “host strain” or “host cell” is an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

The term “expression” refers to the process by which a polypeptide is produced based on a nucleic acid sequence. The process includes both transcription and translation.

The term “end product” refers to an alcohol such as ethanol, or a biochemical selected from the group consisting of an amino acid, an organic acid, citric acid, lactic acid, succinic acid, monosodium glutamate, gluconic acid, sodium gluconate, calcium gluconate, potassium gluconate, glucono delta-lactone, sodium erythorbate, omega 3 fatty acid, butanol, lysine, itaconic acid, 1,3-propanediol, biodiesel, and isoprene

“Biologically active” refer to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein.

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

The terms “thermally stable”, “thermostable” and “thermostability,” with reference to an enzyme, refer to the ability of the enzyme to retain activity after exposure to an elevated temperature. The thennostability of an enzyme, such as an amylase enzyme, is measured by its half-life (t_{1/2}) given in minutes, hours, or days, during which half the enzyme activity is lost under defined conditions. The half-life may be calculated by measuring residual amylase activity for example following exposure to (i.e., challenge by) an elevated temperature. The terms “thermally stable” and “thermostable” mean that at least about 50%, 55%, 60%, 65%, 70%, 75%, 80%, 85%, 90%, 95%, 96%, 97% or 98% of the enzyme that was present/active in the additive before heating to the specified temperature is still present/active after it cools to room temperature. Preferably, at least about 80% of the enzyme that is present and active in the additive before heating to the specified temperature is still present and active after it cools to room temperature.

A “pH range,” with reference to an enzyme, refers to the range of pH values under which the enzyme exhibits catalytic activity.

The terms “pH stable” and “pH stability,” with reference to an enzyme, relate to the ability of the enzyme to retain activity over a wide range of pH values for a predetermined period of time (e.g., 15 min., 30 min., 1 hour).

The phrase “simultaneous saccharification and fermentation (SSF)” refers to a process in the production of biochemicals in which a microbial organism, such as an ethanologenic microorganism, and at least one enzyme, such as an amylase, are present during the same process step. SSF includes the contemporaneous hydrolysis of starch substrates (granular, liquefied, or solubilized) to saccharides, including glucose, and the fermentation of the saccharides into alcohol or other biochemical or biomaterial in the same reactor vessel.

A “slurry” is an aqueous mixture containing insoluble starch granules in water.

The term “total sugar content” refers to the total soluble sugar content present in a starch composition including monosaccharides, oligosaccharides and polysaccharides.

The term “dry solids” (ds) refer to dry solids dissolved in water, dry solids dispersed in water or a combination of both. Dry solids thus include granular starch, and its hydrolysis products, including glucose.

“Dry solid content” refers to the percentage of dry solids both dissolved and dispersed as a percentage by weight with respect to the water in which the dry solids are dispersed and/or dissolved. The initial dry solid content of starch is the weight of granular starch corrected for moisture content over the weight of granular starch plus weight of water. Subsequent dry solid content can be determined from the initial content adjusted for any water added or lost and for chemical gain. Subsequent dissolved dry solid content can be measured from refractive index as indicated below. 8

The term “high DS” refers to aqueous starch slurry with a dry solid content of 34% (wt/wt) or greater.

“Dry substance starch” refers to the dry starch content of a substrate, such as a starch slurry, and can be determined by subtracting from the mass of the substrate any contribution of non-starch components such as protein, fiber, and water. For example, if a granular starch slurry has a water content of 20% (wt/wt), and a protein content of 1% (wt/wt), then 100 kg of granular starch has a dry starch content of 79 kg. Dry substance starch can be used in determining how many units of enzymes to use.

“Liquefact” refers to the product of cooking (heating) and liquefaction (reduction of viscosity) of a starch or starch containing grain slurry (mash).

“Liquefaction” or “liquefy” refers to a process by which starch (or starch containing grains) is/are converted to shorter chain and less viscous dextrins.

“Degree of polymerization (DP)” refers to the number (n) of anhydroglucopyranose units in a given saccharide. Examples of DP1 are the monosaccharides, such as glucose and fructose. Examples of DP2 are the disaccharides, such as maltose, isomaltose and sucrose. Examples of DP3 are the trisaccharides, such as isomaltotriose and panose. DP3+ denotes polymers with a degree of polymerization of greater than 3.

The term “soluble starch substrate” refers to starch that is capable of dissolving in hot water.

The term “glucose syrup” refers to a syrup made from the hydrolysis of starch.

The term “contacting” refers to the placing of referenced components (including but not limited to enzymes, substrates, and fermenting organisms) in sufficiently close proximity to affect an expect result, such as the enzyme acting on the substrate or the fermenting organism fermenting a substrate. Those skilled in the art will recognize that mixing solutions can bring about “contacting.”

An “ethanologenic microorganism” refers to a microorganism with the ability to convert a sugar or other carbohydrates to ethanol.

The term “biochemicals” refers to a metabolite of a microorganism, such as citric acid, lactic acid, succinic acid, monosodium glutamate, gluconic acid, sodium gluconate, calcium gluconate, potassium gluconate, glucono delta-lactone, sodium erythorbate, omega 3 fatty acid, butanol, iso-butanol, an amino acid, lysine, itaconic acid, other organic acids, 1,3-propanediol, vitamins, or isoprene or other biomaterial.

The term “about” refers to +15% to the referenced value.

The following abbreviations/acronyms have the following meanings unless otherwise specified:
EC enzyme commission
CAZy carbohydrate active enzyme
w/v weight/volume
w/w weight/weight
v/v volume/volume
wt % weight percent
° C. degrees Centigrade
g or gm gram
g microgram
mg milligram
kg kilogram
μL and μl microliter
mL and ml milliliter
mm millimeter
μm micrometer
mol mole
mmol millimole
M molar
mM millimolar
μM micromolar
nm nanometer
U unit
ppm parts per million
hr and h hour

### Glucoamylases and Methods of Use, Thereof

In a first aspect, the present invention relates to a method for saccharifying a starch substrate, comprising contacting the substrate with a glucoamylase having at least two times more activity on an α-1,6 bond-containing substrate compared to the glucoamylase from Aspergillus niger under equivalent conditions, wherein the saccharifying with the glucoamylase produces a glucose syrup having a higher level of DP1 compared to saccharifying the same starch substrate with the glucoamylase from Aspergilli niger under equivalent conditions.

In some embodiments, the glucoamylases in the present invention are capable of hydrolyzing α-1,4-glucosidic bonds (linear) as well as α-1, 6-glucosidic bonds (branching). Exemplary substrates containing alpha-1,6-glucosidic bonds are amylopectin, isomaltose, pullulan and panose.

In some embodiments, the glucoamylases in the present invention having at least about two times (e.g., at least about three times, at least about four times, at least about five times, at least about six times, at least about seven times, at least about eight times, at least about nine times, at least about ten times, at least about eleven times, at least about twelve times, at least about thirteen times, at least about fourteen times, at least about fifteen times or a higher ratio) more activity on an α-1,6 bond-containing substrate compared to the glucoamylase from Aspergillus niger under equivalent conditions.

In some embodiments, the glucoamylases in the present invention having at least two times (e.g., at least about three times, at least about four times, at least about five times, at least about six times, at least about seven times, at least about eight times, at least about nine times, at least about ten times or a higher ratio) more activity on a pullulan, panose or isomaltose substrate compared to the glucoamylase from Aspergillus niger under equivalent conditions.

In some embodiments, the glucoamylases comprise an amino acid sequence having preferably at least 80%, at least 83%, at least 85%, at least 90%, at least 92%, at least 93%, at least 94%, at least 95%, at least 96%, at least 97%, at least 98%, and even at least 99%, amino acid sequence identity to the polypeptide of SEQ ID NO: 2, SEQ ID NO: 4, or SEQ ID NO: 6, and having glucoamylase activity.

In some embodiments, the polypeptides of the present glucoamylases are the homologous polypeptides comprising amino acid sequences differ by ten amino acids, preferably by nine amino acids, preferably by eight amino acids, preferably by seven amino acids, preferably by six amino acids, preferably by five amino acids, more preferably by four amino acids, even more preferably by three amino acids, most preferably by two amino acids, and even most preferably by one amino acid from the polypeptide of SEQ ID NO: 2, SEQ ID NO: 4, or SEQ ID NO: 6.

In some embodiments, the polypeptides of the present invention are the variants of polypeptide of SEQ ID NO: 2, SEQ ID NO: 4, or SEQ ID NO: 6 or a fragment thereof having glucoamylase activity.

In some embodiments, the polypeptides of the present invention are the catalytic region comprising the amino acids 21-481 of SEQ ID NO: 2, the amino acids 29-491 of SEQ ID NO: 4, or the amino acids 27-485 of SEQ ID NO: 6, predicted by ClustalX https://www.ncbi.nlm.nih.gov/pubmed/17846036.

In some embodiments, the polypeptides of the present invention are the catalytic region and linker region comprising the amino acids 21-503 of SEQ ID NO: 2, the amino acids 29-513 of SEQ ID NO: 4, or the amino acids 27-506 of SEQ ID NO: 6, predicted by ClustalX https://www.ncbi.nlm.nih.gov/pubmed/17846036.

In some embodiments, the polypeptides of the present invention have maximum activity at a temperature of about 70° C., have over 70% of maximum activity at a temperature of about 62° C. to a temperature of about 77° C., measured at a pH of 5.0, as determined by the assays described, herein. Exemplary temperature ranges for use of the enzyme are 50-85° C., 55-80° C., 55-75° C., and 60-75° C.

In some embodiments, the polypeptides of the present invention are thermostable and retain glucoamylase activity at increased temperature. The polypeptides of the present invention have shown thermostability at pH values ranging from about 4.0 to about 6.0 (e.g., about 4.5 to about 6.0, about 4.0 to about 5.5, about 4.5 to about 5.5, etc.). For example, at pH of about 5.0, the polypeptides of the present invention retain most of glucoamylase activity for an extended period of time at high temperature (e.g., at least 50° C., at least 55° C., at least 60° C., at least 65° C., at least 70° C., at least 75° C., at least 80° C. or a higher temperature). For example, the polypeptides of the present invention retain at least about 45% (e.g., at least about 50%, at least about 55%, at least about 60%, at least about 65%, at least about 70% or a higher percentage) of glucoamylase activity at increased temperature at a pH of from about 4.0 to about 6.0 for at least 1 hour, 2 hours, 3 hours or even longer.

In some embodiments, the polypeptides of the present invention have maximum activity at a pH of about 5, have over 90% of maximum activity at a pH of about 3.5 to a pH of about 6.0, and have over 70% of maximum activity at a pH of about 2.5 to a pH of about 7.0, measured at a temperature of 50° C., as determined by the assays described, herein. Exemplary pH ranges for use of the enzyme are pH 2.5-7.0, 3.0-7.0, 3.5-7.0, 2.5-6.0, 3.0-6.0 and 3.5-6.0.

In some embodiments, the polypeptides of the present invention are low pH stable and retain glucoamylase activity at low pH. The polypeptides of the present invention have shown low pH stability at pH values ranging from about 2.0 to about 7.0 (e.g., about 2.0 to about 6.0, about 2.0 to about 5.0, about 2.0 to about 4.0, etc.). For example, at pH 2.5 to about 6.0, the polypeptides of the present invention retain most of glucogenic activity for an extended period of time at high temperature (e.g. at least 40° C., at least 50° C., at least 55° C., at least 60° C., at least 65° C., at least 70° C. or a higher temperature), and for example, for at least 4 hours, at least 17 hours, at least 24 hours, at least 48 hours, at least 72 hours, or even longer.

In some embodiments, the polypeptides of the present invention have better saccharification performance in comparison with (a glucoamylase from Aspergillus niger) AnGA, at a pH of about 4, or at a pH of about 4.5, or at a pH of about 5, at a temperature range from about 55 to about 75° C., (e.g., about 55° C. to about 70° C., about 60° C. to about 75° C., about 60° C. to about 70° C. etc.) with incubation time for at least 24 hours, at least 48 hours, at least 72 hours, or even longer.

In some embodiments, the polypeptides of the present invention can be used in simultaneous saccharification and fermentation (SSF) process in comparison with the current commercial available glucoamylase products, at a pH of about 3, or at a pH of about 4, or at a pH of about 5, at a temperature range from about 30° C. to about 70° C., (e.g., about 30° C. to about 60° C., about 30° C. to about 50° C., etc.) with incubation time for at least 17 hours, at least 24 hours, at least 48 hours, at least 72 hours, or even longer.

In some embodiments, the polypeptides having glucoamylase activity can be obtained from any of: a Trichoderma sp., an Aspergillus sp., a Humicola sp., a Penicillium sp., a Talaromyces sp., a Symbiotaphrina sp, or a Schizosaccharomyces sp. In one embodiment, the polypeptide having glucoamylase activity is from Penicillium glabrum. In one embodiment, the polypeptide having glucoamylase activity is from Symbiotaphrina kochii. In one embodiment, the polypeptide having glucoamylase activity is from Penicillium brasilianum.

In a second aspect, the present glucoamylases comprise conservative substitution of one or several amino acid residues relative to the amino acid sequence of SEQ ID NO: 2, SEQ ID NO: 4, or SEQ ID NO: 6. Conservative amino acid substitutions are well known in the art.

In some embodiments, the present glucoamylase comprises a deletion, substitution, insertion, or addition of one or a few amino acid residues relative to the amino acid sequence of SEQ ID NO: 2, SEQ ID NO: 4, or SEQ ID NO: 6 or a homologous sequence thereof. In some embodiments, the present glucoamylases are derived from the amino acid sequence of SEQ ID NO: 2, SEQ ID NO: 4, or SEQ ID NO: 6 by conservative substitution of one or several amino acid residues. In some embodiments, the present glucoamylases are derived from the amino acid sequence of SEQ ID NO: 2, SEQ ID NO: 4, or SEQ ID NO: 6 by deletion, substitution, insertion, or addition of one or a few amino acid residues relative to the amino acid sequence of SEQ ID NO: 2, SEQ ID NO: 4, or SEQ ID NO: 6. In all cases, the expression “one or a few amino acid residues” refers to 10 or less, i.e., 1, 2, 3, 4, 5, 6, 7, 8, 9, or 10, amino acid residues. The amino acid substitutions, deletions and/or insertions of SEQ ID NO: 2, SEQ ID NO: 4, or SEQ ID NO: 6 can be at most 10, preferably at most 9, more preferably at most 8, more preferably at most 7, more preferably at most 6, more preferably at most 5, more preferably at most 4, even more preferably at most 3, most preferably at most 2, and even most preferably at most 1.

Alternatively, the amino acid changes are of such a nature that the physicochemical properties of the polypeptides are altered. For example, amino acid changes may improve the thermal stability of the polypeptide, alter the substrate specificity, change the pH optimum, and the like.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

The glucoamylase may be a “chimeric” or “hybrid” polypeptide, in that it includes at least a portion from a first glucoamylase, and at least a portion from a second amylase, glucoamylase, beta-amylase, alpha-glucosidase or other starch degrading enzymes, or even other glycosyl hydrolases, such as, without limitation, cellulases, hemicellulases, etc. (including such chimeric amylases that have recently been “rediscovered” as domain-swap amylases). The present glucoamylases may further include heterologous signal sequence, an epitope to allow tracking or purification, or the like.

The present glucoamylases can be produced in host cells, for example, by secretion or intracellular expression. A cultured cell material (e.g., a whole-cell broth) comprising a glucoamylase can be obtained following secretion of the glucoamylase into the cell medium. Optionally, the glucoamylase can be isolated from the host cells, or even isolated from the cell broth, depending on the desired purity of the final glucoamylase. A gene encoding a glucoamylase can be cloned and expressed according to methods well known in the art. Suitable host cells include bacterial, fungal (including yeast and filamentous fungi), and plant cells (including algae). Particularly useful host cells include Aspergillus niger, Aspergillus oryzae, Trichoderma reesi or Myceliopthora thermophila. Other host cells include bacterial cells, e.g., Bacillus subtilis or B. licheniformis, as well as Streptomyces.

Additionally, the host may express one or more accessory enzymes, proteins, peptides. These may benefit liquefaction, saccharification, fermentation, SSF, and downstream processes. Furthermore, the host cell may produce ethanol and other biochemicals or biomaterials in addition to enzymes used to digest the various feedstock(s). Such host cells may be useful for fermentation or simultaneous saccharification and fermentation processes to reduce or eliminate the need to add enzymes.

The present invention also relates to compositions comprising a polypeptide of the present invention. In some embodiments, a polypeptide comprising an amino acid sequence having preferably at least 80%, at least 83%, at least 85%, at least 90%, at least 92%, at least 93%, at least 94%, at least 95%, at least 96%, at least 97%, at least 98%, and even at least 99%, amino acid sequence identity to the polypeptide of SEQ ID NO: 2, SEQ ID NO: 4, or SEQ ID NO: 6 and having glucoamylase activity can also be used in the enzyme composition. Preferably, the compositions are formulated to provide desirable characteristics such as low color, low odor and acceptable storage stability.

The composition may comprise a polypeptide of the present invention as the major enzymatic component, e.g., a mono-component composition. Alternatively, the composition may comprise multiple enzymatic activities, such as an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, alpha-glucosidase, beta-glucosidase, beta-amylase, isoamylase, haloperoxidase, invertase, laccase, lipase, lysozyme, mannosidase, oxidase, pectinolytic enzyme, peptidoglutaminase, peroxidase, phytase, polyphenoloxidase, proteolytic enzyme, pullulanase, ribonuclease, transglutaminase, xylanase or a combination thereof, which may be added in effective amounts well known to the person skilled in the art.

The polypeptide compositions may be prepared in accordance with methods known in the art and may be in the form of a liquid or a dry composition. For instance, the compositions comprising the present glucoamylases may be aqueous or non-aqueous formulations, granules, powders, gels, slurries, pastes, etc., which may further comprise any one or more of the additional enzymes listed herein, along with buffers, salts, preservatives, water, co-solvents, surfactants, and the like. Such compositions may work in combination with endogenous enzymes or other ingredients already present in a slurry, water bath, washing machine, food or drink product, etc., for example, endogenous plant (including algal or fungal) enzymes, residual enzymes from a prior processing step, and the like. The polypeptide to be included in the composition may be stabilized in accordance with methods known in the art.

The composition may be cells expressing the polypeptide, including cells capable of producing a product from fermentation. Such cells may be provided in a cream or in dry form along with suitable stabilizers. Such cells may further express additional polypeptides, such as those mentioned, above.

Examples are given below of preferred uses of the polypeptides or compositions of the invention. The dosage of the polypeptide composition of the invention and other conditions under which the composition is used may be determined on the basis of methods known in the art.

The present invention is also directed to use of a polypeptide or composition of the present invention in a liquefaction, a saccharification and/or a fermentation process. The polypeptide or composition may be used in a single process, for example, in a liquefaction process, a saccharification process, or a fermentation process. The polypeptide or composition may also be used in a combination of processes for example in a liquefaction and saccharification process, in a liquefaction and fermentation process, or in a saccharification and fermentation process, preferably in relation to starch conversion.

The liquefied starch may be saccharified into a syrup rich in lower DP (e.g., DP1+DP2) saccharides, using alpha-amylases and glucoamylases, optionally in the presence of another enzyme(s). The exact composition of the products of saccharification depends on the combination of enzymes used, the composition of the liquefied starch, the conditions of the saccharification, as well as the type of starch processed. Advantageously, the syrup obtainable using the provided glucoamylases may contain a weight percent of DP3+ of the total oligosaccharides in the saccharified starch below 20%, e.g., 1%, 5%, 10% or 20%. The weight percent of DP1 in the saccharified starch may exceed about 80%, e.g., 75% 85% or 80% 90% or 80%-95%.

Whereas liquefaction is generally run as a continuous process, saccharification is often conducted as a batch process. Saccharification conditions are dependent upon the nature of the liquefact and type of enzymes available. In some cases, a saccharification process may involve temperatures of about 60-90° C. and a pH of about 2.0-4.5, for example, about 2.0, 2.2, 2.4, 2.6, 2.8, 3.0, 3.2, 3.4, 3.6, 3.8, 4.0, 4.2, or 4.4. Saccharification may be performed, for example, at a temperature between about 40° C., about 50° C., or about 55° C. to about 60° C., or about 65° C., or about 70° C., or about 75° C., or about 80° C., or about 85° C., or about 90° C. or higher, in many cases necessitating cooling of the liquefact. By conducting the saccharification process at higher temperatures, the process can be carried out in a shorter period of time or alternatively the process can be carried out using lower enzyme dosage. Furthermore, the risk of microbial contamination is reduced when carrying the liquefaction and/or saccharification process at higher temperature.

Saccharification is normally conducted in stirred tanks, which may take several hours to fill or empty. Enzymes typically are added either at a fixed ratio to dried solids, as the tanks are filled, or added as a single dose at the commencement of the filling stage. A saccharification reaction to make a syrup typically is run over about 24-72 hours, for example, 24-48 hours.

However, it is common only to do a pre-saccharification of typically 40-90 minutes at a temperature between 30-65° C., typically about 60° C., followed by complete saccharification in a simultaneous saccharification and fermentation (SSF). In one embodiment, a process of the invention includes pre-saccharifying starch-containing material before simultaneous saccharification and fermentation (SSF) process. The pre-saccharification can be carried out at a high temperature (for example, 50-85° C., preferably 60-75° C.) before moving into SSF.

In a preferred aspect of the present invention, the liquefaction and/or saccharification includes sequentially or simultaneously performed liquefaction and saccharification processes.

The soluble starch hydrolysate, particularly a glucose rich syrup, can be fermented by contacting the starch hydrolysate with a fermenting organism typically at a temperature around 32° C., such as from 30° C. to 35° C. “Fermenting organism” refers to any organism, including bacterial and fungal organisms, suitable for use in a fermentation process and capable of producing desired a fermentation product. Especially suitable fermenting organisms are able to ferment, i.e., convert, sugars, such as glucose or maltose, directly or indirectly into the desired fermentation product. Examples of fermenting organisms include yeast, such as Saccharomyces cerevisiae and bacteria, e.g., Zymomonas mobilis, expressing alcohol dehydrogenase and pyruvate decarboxylase. The ethanologenic microorganism can express xylose reductase and xylitol dehydrogenase, which convert xylose to xylulose. Improved strains of ethanologenic microorganisms, which can withstand higher temperatures, for example, are known in the art and can be used. See Liu et al. (2011) Sheng Wu Gong Cheng Xue Bao 27:1049-56. Commercially available yeast includes, e.g., Red Star™/Lesaffre Ethanol Red (available from Red Star/Lesaffre, USA) FALI (available from Fleischmann's Yeast, a division of Burns Philp Food Inc., USA), SUPERSTART (available from Alltech), GERT STRAND (available from Gert Strand AB, Sweden), SYNERXIA® ADY (available from DuPont), SYNERXIA®, THRIVE (available from DuPont), FERMIOL (available from DSM Specialties). The temperature and pH of the fermentation will depend upon the fermenting organism. Microorganisms that produce other metabolites, such as citric acid and lactic acid, by fermentation are also known in the art. See, e.g., Papagianni (2007) Biotechnol. Acv. 25:244-63; John et al. (2009) Biotechnol. Acv. 27:145-52.

The saccharification and fermentation processes may be carried out as an SSF process. An SSF process can be conducted with fungal cells that express and secrete glucoamylase continuously throughout SSF. The fungal cells expressing glucoamylase also can be the fermenting microorganism, e.g., an ethanologenic microorganism. Ethanol production thus can be carried out using a fungal cell that expresses sufficient glucoamylase so that less or no enzyme has to be added exogenously. The fungal host cell can be from an appropriately engineered fungal strain. Fungal host cells that express and secrete other enzymes, in addition to glucoamylase, also can be used. Such cells may express amylase and/or a pullulanase, phytase, alpha-glucosidase, isoamylase, beta-amylase cellulase, xylanase, other hemicellulases, protease, beta-glucosidase, pectinase, esterase, redox enzymes, transferase, or other enzymes. Fermentation may be followed by subsequent recovery of ethanol.

In accordance with the present invention the fermentation includes, without limitation, fermentation processes used to produce alcohols (e.g., arabinitol, butanol, ethanol, glycerol, methanol, ethylene glycol, propylene glycol, butanediol, glycerin, sorbitol, and xylitol); organic acids (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); ketones (e.g., acetone); amino acids (e.g., aspartic acid, glutamic acid, glycine, lysine, serine, and threonine); an alkane (e.g., pentane, hexane, heptane, octane, nonane, decane, undecane, and dodecane); a cycloalkane (e.g., cyclopentane, cyclohexane, cycloheptane, and cyclooctane); an alkene (e.g. pentene, hexene, heptene, and octene); gases (e.g., methane, hydrogen (H₂), carbon dioxide (CO₂), and carbon monoxide (CO)); antibiotics (e.g., penicillin and tetracycline); enzymes; vitamins (e.g., riboflavin, B₁₂, beta-carotene); and hormones. In a preferred aspect, the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry.

In such preferred embodiment, the process is typically carried at a temperature between 28° C. and 36° C., such as between 29° C. and 35° C., such as between 30° C. and 34° C., such as around 32° C., at a pH in the range between 3 and 7, preferably from pH 3.5 to 6, or more preferably from pH 4 to 5.

The present invention provides a use of the glucoamylase of the invention for producing glucoses and the like from raw starch or granular starch. Generally, glucoamylase of the present invention either alone or in the presence of an alpha-amylase can be used in raw starch hydrolysis (RSH) or granular starch hydrolysis (GSH) process for producing desired sugars and fermentation products. The granular starch is solubilized by enzymatic hydrolysis below the gelatinization temperature. Such “low-temperature” systems (known also as “no-cook” or “cold-cook”) have been reported to be able to process higher concentrations of dry solids than conventional systems (e.g., up to 45%).

A “raw starch hydrolysis” process (RSH) differs from conventional starch treatment processes, including sequentially or simultaneously saccharifying and fermenting granular starch at or below the gelatinization temperature of the starch substrate typically in the presence of at least an glucoamylase and/or amylase. Starch heated in water begins to gelatinize between 50° C. and 75° C., the exact temperature of gelatinization depends on the specific starch. For example, the gelatinization temperature may vary according to the plant species, to the particular variety of the plant species as well as with the growth conditions. In the context of this invention the gelatinization temperature of a given starch is the temperature at which birefringence is lost in 5% of the starch granules using the method described by Gorinstein. S. and Lii. C., Starch/Starke, Vol. 44 (12) pp. 461-466 (1992).

The glucoamylase of the invention may also be used in combination with an enzyme that hydrolyzes only alpha-(1, 6)-glucosidic bonds in molecules comprising at least four glucosyl residues. Preferably, the glucoamylase of the invention is used in combination with pullulanase or isoamylase. The use of isoamylase and pullulanase for debranching of starch, the molecular properties of the enzymes, and the potential use of the enzymes together with glucoamylase is described in G. M. A. van Beynum et al., Starch Conversion Technology, Marcel Dekker, New York, 1985, 101-142.

Processes for making beer are well known in the art. See, e.g., Wolfgang Kunze (2004) “Technology Brewing and Malting,” Research and Teaching Institute of Brewing, Berlin (VLB), 3rd edition. Briefly, the process involves: (a) preparing a mash, (b) filtering the mash to prepare a wort, and (c) fermenting the wort to obtain a fermented beverage, such as beer. Preferred beer types comprise ales, stouts, porters, lagers, bitters, malt liquors, high-alcohol beer, low-alcohol beer, low-calorie beer or light beer. Preferred fermentation processes used include alcohol fermentation processes, which are well known in the art. Preferred fermentation processes are anaerobic fermentation processes, which are well known in the art.

The brewing composition comprising a glucoamylase, in combination with an amylase and optionally a pullulanase and/or isoamylase, may be added to the mash of step (a) above, i.e., during the preparation of the mash. Alternatively, or in addition, the brewing composition may be added to the mash of step (b) above, i.e., during the filtration of the mash. Alternatively, or in addition, the brewing composition may be added to the wort of step (c) above, i.e., during the fermenting of the wort.

### Examples

Unless defined otherwise herein, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used with this disclosure.

The disclosure is further defined in the following Examples. It should be understood that the Examples, while indicating certain embodiments, is given by way of illustration only. From the above discussion and the Examples, one skilled in the art can ascertain essential characteristics of this disclosure, and without departing from the spirit and scope thereof, can make various changes and modifications to adapt to various uses and conditions.

### Example 1. Expression of Glucoamylases

The nucleic acid sequence for the Penicillum glabrum glucoamylase (PglGA1) gene, and the amino acid sequence of the hypothetical protein encoded by the PglGA1 gene were found in the JGI database (scaffold_7:734096-736255, protein ID: 389044, https://genome.jgi.doe.gov/cgi-bin/dispGeneModel?db=Pengl1&id=389044). A codon modified synthetic DNA sequence encoding full-length PglGA1 was synthesized and inserted into the pTTT expression vector (described in published PCT Application WO2011/063308).

The nucleotide sequence of the PglGA1 synthetic gene used for expression is set forth below as SEQ ID NO: 1

ATGCGATCTACCTTCCTCACCGTTGCTGGCGTCCTCGTTGGCGGCCAGGT
TGCTGCCGCTAACCCTTTCPACTCTCTGGACTCCTTCATTCTGAAGGAAG
GCGCCCGATCTTACCAGGGCATCATTGACAACCTGGGCAACAAGGGCGTC
AAGGCTCCCGGCACCGCCGCTGGTCTGTTCGTCGCCTCACCCAACACCGC
TAACCCCGACTACTTCTACACCTGGACCCGCGACTCTGCTCTGACCTTCA
AGTGCCTGATTGACCTGTTTGACGGCGGCTCCACCGAGTTCGGCCTGAAG
AACTCTGAGCTGGAGACCGACATCCGAAACTACGTTTCTAGCCAGGCTGT
TCTGCAGAACGTTAGCAACCCTAGCGGCACCCTAGAGGACGGCACCGGCC
TCGGCGAGCCTAAGTTTGAAGTTGACCTGAACCCTTTCACCGGCTCTTGG
GGCCGCCCTCAGCGAGACGGCCCCGCTCTCCGCGCCACCGCTCTCATTAC
CTACACCAACTACCTCCTGTCTCAGGGCCAGAAGAGCGAGGCCGTCAACA
TCATGTGGCCCATTATTTCTAACGACCTCGCTTACGTTGGCCAGTACTGG
AACGACACCGGCTTTGACCTGTGGGAAGAGACCGACGGCTCTAGCTTCTT
CACCCTCGCCGTTCAGCACCGCGCCCTGGTTCAGGGCGCCACCCTCGCTC
AGAAGCTGGGCAAGTCTTGCGCTGCTTGCAGCTCTCAGGCTCCTCAGATT
CTGTGCTTCCTGCAGTCTTTCTGGAACGGCAAGTACATCACCGCTAACAT
TAACCTGGACACCAGCCGAACCGGCATTGACGCCAACACCCTCCTGGGCA
GCATCCACACCTTTGACCCCGAGGCTGCTTGCGACGACTCTACCTTCCAG
CCTTGCAGCGCCCGAGCTCTCGCTAACCACAAGGTTTACGTTGACGCTTT
CCGATCTATCTACAAGATTAACTCCGGCATTGCTGAGGGCTCTCCCGCCA
ACGTTGGCCGATACCCCGAGGACGTTTACCAGGGCGGCAACCCTTGGTAT
CTGACCACCCTCGCGTCTGCTGAGCTGCTGTACGACGCTCTGTACCAGTG
GAACAAGATTGGCGGCTTGGACGTTACCGAGACCAGCCTCGCTTTCTTCA
AGGACTTCCACAGCTCCGTTAAGACCGGCAGCTACTCTGCCCACTCCCAG
ACCTACAAGACCCTGACCAGCGCCATAAGGACCTACGCTGACGGCTTCGT
TGGCCTCGTCCAGAAGTACACCCCCGCTAACGGCTCTCTCGCTGAGCAGT
ACAACCGAAACACCAGCGTCCCTCTGTCCGCCAACGACCTGACCTGGTCT
TTCGCTTCTTTCCTCACCGCTATTCAGCGACGAGAGTCTATTGTTCCCGG
CTCTTGGGGCGAGAAGTCTGCCAACACCGTCCCTACCACCTGCAGCGCTT
CTCCCGTTACCGGCACCTACAAGGCTGCTACCAGCACCTTCCCCACCAGC
ACCGCTGGCTGCGTCCCTGCTACCGACGTCGTCCCCATTACCTTCTACCT
CATTGAGAACACCTACTACGGCGAGAACGTTTACATGACCGGCAACATTA
GCGCCCTGGGCAACTGGGACACCAGCGACGGCCTCGCCCTGGACGCCGGA
CTGTACACCGAGACCGACAACCTGTGGTTCGGCACCCTTGAACTGGTTAC
CGCTGGCACCCCTTTTGAATACAAGTACTACAAGATTGAGCCTAACGGCA
CCGTTACCTGGGAGTCTGGCGACAACCGCGTCTCCGTTGTTCCTACCGGC
TGCCCCATCCAGCCTAGCCTCCACGACGTTTGGCGATCCTAA

The nucleic acid sequence for the Symbiotaphrina kochii CBS 250.77 glucoamylase (SkoGA1) gene, and the amino acid sequence of the hypothetical protein encoded by the SkoGA1 gene were found in the JGI database (scaffold 6:771037-773008, protein ID: 779991, https://genome.jgi.doe.gov/cgi-bin/dispGeneModel?db=Symkol&id=779991). A codon modified synthetic DNA sequence encoding full-length SkoGA1 was synthesized and inserted into the pTTT expression vector (described in published PCT Application WO20111/063308).

The nucleotide sequence of the SkoGA1 synthetic gene used for expression is set forth below as SEQ ID NO: 3

ATGTGGGCTGTTAACGCTGCTTTCGCGGGCGTTGCTTCCATTCTCCTGGG
CCCCGCGTCCGTTTTCCACCGATGGCAGGACCGATCTACCTCCCAGGCAA
GCACCCTGGACTCTTACCTCACCAGCGAGGCTTCTCTGTCTTACCAGGGC
ATTCTGAACAACCTGGGCGACACCGGCTCCAAGGCTCCCGGCACCGCTGC
TGGCCTCCTGGTTGCGAGCCCTAACACCGCCAACCCCGACTACTTCTACT
CTTGGACCCGAGACTCCGCTCTCACCTTCAAGTGCCTCATTGACCTGTAC
ATTAGCGGCAACACCACCCTGGACATTAACTACACCACCCTGCAGACCGA
CATTGAGAACTACATTAGCGCCCAGGCCGTCCTGCAGAACGTTTCTAACC
CTAGCGGCACCCTCGCTACCGGCGCGGGTCTCGGCGAGCCCAAGTTTGAG
GTTGACCTCAACCCTTTCAGCGGCTCTTGGGGCCGCCCCCAGCGCGACGG
CCCCGCCTTGCGAGCCACCGCTCTGATTGCTTACTCCCGATGGCTGGTTA
GCAACGACCAGTCCTCTGTTGCTGCCGACACCATTTGGCCTATTCTCGCC
AACGACCTCGCTTACGTCGCCGAGTACTGGAACCAGACCGGCTTTGATCT
GTGGGAAGAGATTGAGGGCAGCTCTTTCTTCACCGTTGCTGTTCAGCACC
GAGCTCTCGTGGAGGGCGCGTCTATTGCTTCCACCCTGGGCAAGTCTTGC
GACGCTTGCACCTCTCAGGCTCCTCAGATTCTGTGCTTCCTGCAGTCTTT
CTGGAACGGCGACTACATTACCGCCAACATCAACGTAGATGACGGCCGAA
GCGGCATTGACGCCAACACCATCCTCGGCACCATCCACACCTTTGATCCT
TCTGCCGCTTGCGACGACTCTACCTTCCAGCCTTGCAGCTCCCGAGCTCT
GGCTAACCACAAGGTTTTCGTTGACGCTTTCCGATCTATATACACCATTA
ACGGCGGCCTGGAAGAAGGCCAGGCTGCCPACGTTGGCCGATACCCCGAG
GACGTTTACCAGGGCGGCAACCCTTGGTATCTGAACACCCTCGCTGCCGC
TGAGCTGCTGTACGACGCCCTGTACCAGTGGTCTAAGATTGGCAGCCTGA
CCGTCACCGACACCAGCCTCGCTTTCTTCCAGGACCTGTCTAGCTCCGTT
GAGCCCGGCACCTACTCTTCCGGCTCTGACACCTTTGAAACCCTGACCAG
CGCCATCCACACCTACGCTGACGGCTTCGTCAGCCTCGTTCAGACCTACA
CCCCTAGCAACGGCAGCCTCGCTGAGCAGTACAACCGAGACACCGGCGTT
CCTCTGTCCGCTAACGACCTCACCTGGTCTTACGCTGCTCTCCTGACCTC
CGTTCAGAGCAGAAGCAGCATCATGCCCGCTTCTTGGGGCGAGCCTAGCG
CCATCGCCGTTCCTTCTACCTGCAGCAGCTCTAGCGTTGCTGGCACCTAC
TCCGTTGTTACCGCTGCTTTCCCCACCAGCACCGCAGGCTGCGTCCCCGC
TATTACCGTCCCCGTTACCTTCTACCTCATTGAGACCACCACCTACGGCG
AGAACGTTTACATGACCGGCGACATCTCTGTTCTCGGCGACTGGTCTACC
AGCTCTGGCTACCCCCTGACCGCCTCGCTGTACACCAGCTCTGAGAACCT
GTGGTTCGCAAGCGTAGAGGGCGTCGCCGCTGGCACCAGCTTTGAGTACA
AGTACTACAAGATAGAATCTGACGGCTCCGTTACCTGGGAGGGCGCCAAC
AACCGAGTTTACACCGTCCCTACCGGCTGCCCTATTCAGCCTCAGGTTCA
CCACGTTTGGCAGACCTGA

The nucleic acid sequence for the Penicillium brasilianum glucoamylase (PbrGA5) gene, and the amino acid sequence of the hypothetical protein encoded by the PbrGA5 gene were found in the NCBI database (NCBI Accession No.: CDHK01000002.1: from 848163 to 850126 (gene) and CEJ55559.1 (protein)). A codon modified synthetic DNA sequence encoding full-length PbrGA5 was synthesized and inserted into the pTTT expression vector (described in published PCT Application WO2011/063308).

The nucleotide sequence of the PbrGA5 synthetic gene used for expression is set forth below as SEQ ID NO: 5

ATGCGCCCTACCCTGTTCACCGGCGTTGCCTCCGTCCTGTGGACCGGCAG
CCTCATTTTCGCTTCTCCTAGCAGCAAGAACGTTGACCTCGCCTCCTTCA
TTAGCAAAGAGGGCCAGCGATCTATTCTCGGCATCACCGAGAACCTGGGC
GGCAAGGGCTCTAAGACCCCCGGCACCGCCGCTGGCCTGTTCATTGCATC
CCCCAACATGGCTAACCCCAACTACTACTACACCTGGACCCGAGACTCTG
CTCTGACCATTAACTGCCTGATTGACCTGTTTGAATCTAGCGGCGGCGGC
TTCTCTACCAGCTCTAAAGAACTGGAGACCGACATCCGAAACTACGTTAG
CGCCCAGGCCGTCCTGCAGAACGTCTCCAACCCTAGCGGCACCCTGCAGG
ACGGCTCCGGCCTGGGCGAGCCTAAGTTTGAGGTTGACCTGAACCCTTTC
TCTGGCTCTTGGGGCCGCCCTCAGCGCGACGGCCCCGCTCTACGGGCCAC
CGCCATGATTACCTACGCTGACTGGCTCATTTCCCACGGCCAGAAGTCTG
AGGCTGCGTCTATTATGTGGCCTATCATTGCTAACGACCTCGCTTACGTC
GGCCAGTACTGGAACAACACCGGCTTTGATCTGTGGGAGGAAGTAGACGG
CAGCTCTTTCTTCACCCTCGCCGTTCAGCACCGAGCTCTCGTCCAGGGCT
CTAGCCTCGCTCAGAAGCTGGGCAAGTCTTGCCCCGCTTGCAAGTCTCAG
GCTCCTCAGATTCTGTGCTTCCTGCAGTCTTTCTGGAACGGCAACTACAT
TACCGCCAACATCAACCTGGACACCAGCCGATCTGGCATTGACCTGAACT
CCATCCTGGGCTCCATCCACACCTTTGATCCCGAGGCTGCTTGCGACGAC
TCCACCTTCCAGCCTTGCAGCGCCCGCGCCCTCGCCAACCACAAGGTTTA
CGTTGACTCTTTCCGATCTATCTACACCATTAACGCTGGCATTGGCAAGG
GCAGCGCTGCTAACGTTGGCCGATACCCCGAGGACGTTTACCAGGGCGGC
AACCCTTGGTATCTCGCCACCCTCGCTGCTGCCGAGATGCTGTACGACGC
TCTGTACCAGTGGAACAAGATTGGCAAGCTGGACGTTACCGACACCAGCC
TCGCTTTCTTCAAGGACTTTGACGCCAGCGTCCGAAAGGGCTCTTACTCC
GCCCACTCTAGCACCTACAAGACCCTCACCAGCGCTATCCGTACCTACGC
TGACGGCTTCCTGACCCTGGTTCAGGAATACACCCCTTCTAACGGCTCTC
TCGCTGAGCAGTACAACCGAAACACCAGCGTCCCTCTGTCTGCCAACGAC
CTCACCTGGTCTTACGCTTCTTTCGTTACCGCCGTCCAGCGACGATCTAG
CATCGTCCCCGCTTCTTGGGGCGAGAAGTCTGCTAACGTTGTTCCCACCA
CCTGCAGCGCGTCCCCCGTTACCGGCACCTACCAGGCCGTTAGCTCCGCT
TTCCCTACCAGCACCGGCTGCGTCCCCGCCACCGACGTCGTTCCTATTAC
CTTCTACCTGATTGAGAACACCTTCTACGGCGAGAACGTTTTCATGACCG
GCAACATCTCCGCCCTCGGCAACTGGGACACCTCCAACGGCTTCCCTCTG
ACCGCTAACCTGTACACCGAGACCAACAACCTGTGGTTCGCAAGTGTTGA
GCTGGTTGCCGCCGGAACTCCTTTTGAATACAAGTACTACAAGGTTGAGC
CTAACGGCACCGTTATTTGGGAGAACGGCGACAACCGAGTTTACGTTGCT
CCTACCGGCTGCCCCATTCAGCCTAACCAGCACGACGTTTGGCGAAGCTA
A

The plasmids encoding the PglGA1, SkoGA1 and PbrGA5 enzyme were transformed into a suitable Trichoderma reesei strain using protoplast transformation (Te'o et al., J. Microbiol. Methods 51:393-99, 2002). The transformants were selected and fermented by the methods described in WO 2016/138315. Supernatants from these cultures were used to confirm the protein expression by SDS-PAGE analysis and assay for enzyme activity.

PglGA1, SkoGA1 and PbrGA5 were purified via the beta-cyclodextrin coupled Sepharose 6 affinity chromatography. Glucoamylase activity assay and SDS-PAGE were performed to determine purity and concentration. The target protein-containing fractions were pooled and concentrated using an Amicon Ultra-15 device with 10 K MWCO. The purified samples were above 90% pure and were stored in 40% glycerol at −80° C. until usage. Protein sequence confirmation for PglGA1, SkoGA1 and PbrGA5 glucoamylases was performed using mass spectroscopy analysis.

The amino acid sequence of the mature form of the PglGA1 is set forth below as SEQ ID NO: 2:

NPFNSLDSFILKEGARSYQGIIDNLGNKGVKAPGTAAGLFVASPNTANPD
YFYTWTRDSALTFKCLIDLFDGGSTEFGLKNSELETDIRNYVSSQAVLQN
VSNPSGTLEDGTGLGEPKFEVDLNPFTGSWGRPQRDGPALRATALITYTN
YLLSQGQKSEAVNIMWPIISNDLAYVGQYWNDTGFDLWEETDGSSFFTLA
VQHRALVQGATLAQKLGKSCAACSSQAPQILCFLQSFWNGKYITANINLD
TSRTGIDANTLLGSIHTFDPEAACDDSTFQPCSARALANHKVYVDAFRSI
YKINSGIAEGSPANVGRYPEDVYQGGNPWYLTTLASAELLYDALYQWNKI
GGLDVTETSLAFFKDFHSSVKTGSYSAHSQTYKTLTSAIRTYADGFVGLV
QKYTPANGSLAEQYNRNTSVPLSANDLTWSFASFLTAIQRRESIVPGSWG
EKSANTVPTTCSASPVTGTYKAATSTFPTSTAGCVPATDVVPITFYLIEN
TYYGENVYMTGNISALGNWDTSDGLALDAGLYTETDNLWFGTLELVTAGT
PFEYKYYKIEPNGTVTWESGDNRVSVVPTGCPIQPSLHDVWRS

The amino acid sequence of the mature form of the SkoGA1 is set forth below as SEQ ID NO: 4:

STSQASTLDSYLTSEASLSYQGILNNLGDTGSKAPGTAAGLLVASPNTAN
PDYFYSWTRDSALTFKCLIDLYISGNTTLDINYTTLQTDIENYISAQAVL
QNVSNPSGTLATGAGLGEPKFEVDLNPFSGSWGRPQRDGPALRATALIAY
SRWLVSNDQSSVAADTIWPILANDLAYVAEYWNQTGFDLWEEIEGSSFFT
VAVQHRALVEGASIASTLGKSCDACTSQAPQILCFLQSFWNGDYITANIN
VDDGRSGIDANTILGTIHTFDPSAACDDSTFQPCSSRALANHKVFVDAFR
SIYTINGGLEEGQAANVGRYPEDVYQGGNPWYLNTLAAAELLYDALYQWS
KIGSLTVTDTSLAFFQDLSSSVEPGTYSSGSDTFETLTSAIHTYADGFVS
LVQTYTPSNGSLAEQYNRDTGVPLSANDLTWSYAALLTSVQSRSSIMPAS
WGEPSAIAVPSTCSSSSVAGTYSVVTAAFPTSTAGCVPAITVPVTFYLIE
TTTYGENVYMTGDISVLGDWSTSSGYPLTASLYTSSENLWFASVEGVAAG
TSFEYKYYKIESDGSVTWEGGNNRVYTVPTGCPIQPQVHDVWQT

The amino acid sequence of the mature form of the PbrGA5 is set forth below as SEQ ID NO: 6:

NVDLASFISKEGQRSILGITENLGGKGSKTPGTAAGLFIASPNMANPNYY
YTWTRDSALTIKCLIDLFESSGGGFSTSSKELETDIRNYVSAQAVLQNVS
NPSGTLQDGSGLGEPKFEVDLNPFSGSWGRPQRDGPALRATAMITYADWL
ISHGQKSEAASIMWPIIANDLAYVGQYWNNTGFDLWEEVDGSSFFTLAVQ
HRALVQGSSLAQKLGKSCPACKSQAPQILCFLQSFWNGNYITANINLDTS
RSGIDLNSILGSIHTFDPEAACDDSTFQPCSARALANHKVYVDSFRSIYT
INAGIGKGSAANVGRYPEDVYQGGNPWYLATLAAAEMLYDALYQWNKIGK
LDVTDTSLAFFKDFDASVRKGSYSAHSSTYKTLTSAIRTYADGFLTLVQE
YTPSNGSLAEQYNRNTSVPLSANDLTWSYASFVTAVQRRSSIVPASWGEK
SANVVPTTCSASPVTGTYQAVSSAFPTSTGCVPATDVVPITFYLIENTFY
GENVFMTGNISALGNWDTSNGFPLTANLYTETNNLWFASVELVAAGTPFE
YKYYKVEPNGTVIWENGDNRVYVAPTGCPIQPNQHDVWRS

### Example 2. Glucoamylase Substrate Specificity

Substrate specificity of glucoamylases PglGA1 (SEQ ID NO: 2), SkoGA1 (SEQ ID NO: 4), PbrGA5 (SEQ ID NO: 6), and AnGA (Aspergillus niger glucoamylase, wildtype, SEQ ID NO: 7) was assayed based on the release of glucose by glucoamylase from the following substrates: soluble starch, corn starch, pullulan, panose, and isomaltose. The coupled glucose oxidase/peroxidase (GOX/HRP) and 2,2′-Azino-bis 3-ethylbenzothiazoline-6-sulfonic acid (ABTS) method (Anal. Biochem. 105 (1980), 389-397) was used as described below.

Substrate solutions were prepared by mixing 9 mL of each substrate mentioned above (1% in water, w/v) and 1 mL of 0.5 M pH 4.5 sodium acetate buffer in a 15-mL conical tube. Coupled enzyme (GOX/HRP) solution with ABTS was prepared in 50 mM sodium acetate buffer (pH 5.0), with the final concentrations of 2.74 mg/mL ABTS, 0.1 U/mL HRP, and 1 U/mL GOX. Glucoamylase samples (5 ppm for soluble starch, 50 ppm for other substrates) were prepared in Milli Q water. Each glucoamylase sample (10 μL) was transferred into a new microtiter plate (Corning 3641) containing 90 μL of substrate solution. The reactions were carried out at 32° C. for 60 min and 60° C. for 30 min, respectively, with shaking (650 rpm) in iEMS incubator (ThermoFisher). The reaction was quenched by adding 50 μL of 0.1 N H₂SO₄. The reaction mixtures (5 μL) were transferred to a 384-well plates (Greiner 781101), followed by the addition of 45 μL of ABTS/GOX/HRP solution. The microtiter plates containing the reaction mixture were measured at 405 nm at 25 seconds intervals for 5 min on SoftMax Pro plate reader (Molecular Device). The output was the reaction rate, Vo, which was directly used to indicate the enzyme activity.

The activity towards different substrates of PglGA1, SkoGA1, PbrGA5 as well as the benchmark AnGA was summarized in Table 1. PglGA1, SkoGA1, and PbrGA5 showed higher activities on all substrates than AnGA under both conditions evaluated: 30 min at 60° C., and 60 min at 32° C. In particular, the tested glucoamylases activities on the substrates having α-1,6 bonds, such as isomaltose, pannose and pullulan substrates, were several fold higher than that of AnGA when tested at 60° C. for 30 min (8.9, 4.1, and 7.9 fold, respectively for PglGA1; 2.7, 2.3, and 6.0 fold, respectively for SkoGA1; 3.6, 2.5, and 6.4 fold, respectively for PbrGA5).

TABLE 1
Substrate specificity of PglGA1, SkoGA1,
and PbrGA5 compared with AnGA

| Incubation | Substrate | AnGA | PglGA1 | SkoGA1 | PbrGA5 |
|---|---|---|---|---|---|
| 60° C., 30 min | Isomaltose | 4.0 | 35.6 | 10.6 | 14.4 |
| | Panose | 68.4 | 283 | 160 | 174 |
| | Pullulan | 42.1 | 331 | 252 | 271 |
| | Soluble starch | 65.8 | 78.3 | 115. | 93.9 |
| | Corn starch | 17.7 | 28.6 | 27.0 | 36.0 |
| 32° C., 60 min | Isomaltose | 1.4 | 21.7 | 4.7 | 6.4 |
| | Panose | 26.7 | 183 | 87.9 | 95.5 |
| | Pullulan | 39.8 | 227 | 196 | 160 |
| | Soluble starch | 28.2 | 33.7 | 52.6 | 46.7 |
| | Corn starch | 20.7 | 22.9 | 19.1 | 24.1 |

### Example 3. pH Effect on Glucoamylase Activity

The effect of pH (from 2.0 to 7.0) on glucoamylase activity was monitored using soluble starch (1% in water, w/w) as substrate. Buffer working solutions consisted of the combination of glycine/sodium acetate/HEPES (250 mM), with pH varying from 2.0 to 7.0. Substrate solutions were prepared by mixing soluble starch (1% in water, w/w) with 250 mM buffer solution at a ratio of 9:1. Enzyme working solutions were prepared in water at a certain dose (showing signal within linear range as per dose response curve). All the incubations were carried out at 50° C. for 10 min. The glucose release was measured by following the same procedure as described above for substrate specificity of glucoamylase. Enzyme activity at each pH was reported as relative activity compared to enzyme activity at optimum pH. The pH profiles of the glucoamylases are shown in Table 2. PglGA1 showed optimal activity at pH 4.0 to 5.0 and its pH range (within which the activity was kept at >70%) was from 2.4 to 6.9. SkoGA1 showed optimal activity at pH 5.0 and its pH range was from 2.6 to 7.0. PbrGA5 showed optimal activity at pH 5.0 and its pH range was from 3.1 to 6.7. PglGA1 and SkoGA1 retained 60% and 50% of their activities at pH 2.0, respectively, while AnGA retained less than 50% at pH 2.

TABLE 2
pH profiles of glucoamylases

Relative activity (%)

| pH | AnGA | PglGA1 | SkoGA1 | PbrGA5 |
|---|---|---|---|---|
| 2.0 | 48 | 60 | 50 | 39 |
| 2.5 | 63 | 73 | 66 | 52 |
| 3.0 | 75 | 87 | 88 | 69 |
| 3.5 | 91 | 92 | 95 | 91 |
| 4.0 | 100 | 100 | 97 | 92 |
| 5.0 | 100 | 100 | 100 | 100 |
| 6.0 | 96 | 90 | 91 | 92 |
| 7.0 | 81 | 67 | 76 | 58 |

### Example 4. Temperature Effect on Glucoamylase Activity

The effect of temperature (evaluated from 40° C. to 90° C.) on glucoamylase activity was monitored using soluble starch (1% in water, w/w) as substrate. Substrate solutions were prepared by mixing 9 mL of soluble starch (1% in water, w/w) and 1 mL of 0.5 M buffer (pH 5.0 sodium acetate) into a 15-mL conical tube. Enzyme working solutions were prepared in water at a certain dose (showing signal within linear range as per dose response curve). Incubations were performed at temperatures from 40° C. to 90° C., respectively, for 10 min. The glucose release was measured by following the same procedure as described above for substrate specificity of glucoamylase. Activity at each temperature was reported as relative activity compared to enzyme activity at optimum temperature. The temperature profiles of glucoamylases are shown in Table 3. PglGA1, SkoGA1, and PbrGA5 all displayed optimal activity at 70° C. Their temperature ranges (within which the activity was kept at >70%) are from 62° C. to 77° C. for PglGA1, from 60° C. to 77° C. for SkoGA1, and from 61° C. to 74° C. for PbrGA5. When the incubation temperature was at 80° C., PglGA1 and SkoGA1 retained greater than 50% of its maximal activity while AnGA lost 90% under the same conditions.

TABLE 3
Temperature profiles of glucoamylases

Relative activity (%)

| Temp. (° C.) | AnGA | PglGA1 | SkoGA1 | PbrGA5 |
|---|---|---|---|---|
| 90 | 5 | 11 | 9 | 7 |
| 80 | 10 | 58 | 54 | 18 |
| 70 | 100 | 100 | 100 | 100 |
| 60 | 78 | 64 | 70 | 68 |
| 55 | 67 | 58 | 54 | 60 |
| 50 | 52 | 44 | 47 | 46 |
| 45 | 43 | 34 | 37 | 42 |
| 40 | 31 | 25 | 27 | 27 |

### Example 5. Evaluation of Glucoamylases on Saccharification at pH 4.5 at Different Temperatures

The saccharification performance of PglGA1, SkoGA1, PbrGA5 and AnGA were evaluated under different incubation temperatures. Alpha-amylase-pretreated corn starch liquefact (prepared at 34% ds, pH 2.9) was used as a starting substrate. The glucoamylases were dosed at 40 μg/gds, which was determined to be a median effective dose (data not shown). The incubations were performed at pH 4.5, at 60, 65 and 69° C., and samples were collected at both 24 and 48 hours to monitor the reactions. All the incubations were quenched by heating at 100° C. for 15 min. Aliquots were removed and diluted 400-fold in 5 mM H₂SO₄ for HPLC analysis using an Agilent 1200 series system with a Phenomenex Rezex-RFQ Fast Fruit column (cat #00D-0223-KO) run at 80° C. 10 μL samples were loaded on the column and separated with an isocratic gradient of 5 mM H₂SO₄ as the mobile phase at a flow rate of 1.0 mL/min. The oligosaccharide products were detected using a refractive index detector, and the standards were run to determine elution times of each sugar of interest (DP3+, DP3, DP2 and DP1). The numbers in Table 4 reflect the peak area percentage of each DP(n) as a fraction of the total DP1 to DP3+. The results of DP1 quantation showed that PglGA1, SkoGA1, and PbrGA5 retained a significant portion of their activity even when the incubation temperature was increased up to 69° C., while AnGA lost more activity as temperature was raised from 60 to 69° C. These data suggest that PglGA1, SkoGA1, and PbrGA5 could be used in higher temperature saccharification processes.

TABLE 4
Sugar compositions results for glucoamylases incubated
at pH 4.5 with corn starch liquefact at 60, 65, 69° C.

| | | Temper- | | | | |
|---|---|---|---|---|---|---|
| Incubation | | ature | | | | |
| time | Enzyme | (° C.) | DP3+% | DP3% | DP2% | DP1% |
| 24 h | AnGA | 60 | 18.9 | 0.8 | 1.6 | 78.7 |
| | | 65 | 24.0 | 0.4 | 4.1 | 71.5 |
| | | 69 | 29.9 | 0.8 | 12.9 | 56.4 |
| | PglGA1 | 60 | 13.0 | 0.9 | 3.1 | 83.0 |
| | | 65 | 13.8 | 0.9 | 3.4 | 81.9 |
| | | 69 | 9.4 | 0.6 | 3.0 | 87.0 |
| | SkoGA1 | 60 | 3.0 | 0.5 | 2.4 | 94.0 |
| | | 65 | 3.3 | 0.6 | 2.7 | 93.4 |
| | | 69 | 2.5 | 0.6 | 3.6 | 93.3 |
| | PbrGA5 | 60 | 6.0 | 0.5 | 1.8 | 91.6 |
| | | 65 | 8.1 | 0.7 | 2.3 | 89.0 |
| | | 69 | 3.8 | 0.5 | 2.4 | 93.4 |
| 48 h | AnGA | 60 | 16.3 | 0.8 | 1.5 | 81.4 |
| | | 65 | 21.0 | 0.8 | 2.4 | 75.8 |
| | | 69 | 29.6 | 0.4 | 12.6 | 57.4 |
| | PglGA1 | 60 | 6.8 | 0.6 | 3.5 | 89.1 |
| | | 65 | 4.3 | 0.6 | 4.1 | 91.0 |
| | | 69 | 4.3 | 0.6 | 4.5 | 90.6 |
| | SkoGA1 | 60 | 2.6 | 0.4 | 3.7 | 93.3 |
| | | 65 | 2.0 | 0.5 | 4.4 | 93.1 |
| | | 69 | 2.5 | 0.7 | 5.4 | 91.4 |
| | PbrGA5 | 60 | 2.7 | 0.5 | 2.9 | 93.8 |
| | | 65 | 2.2 | 0.6 | 3.4 | 93.8 |
| | | 69 | 2.8 | 0.5 | 3.9 | 92.8 |

What is claimed is:

1. A method for saccharification of a starch substrate, comprising contacting the substrate with a glucoamylase having at least two, at least three, or at least four times more activity on an α-1,6 bond-containing substrate compared to the glucoamylase from Aspergillus niger under equivalent conditions, wherein saccharifying with the glucoamylase produces a glucose syrup having a higher level of glucose compared to saccharifying the same starch substrate with the glucoamylase from Aspergillus niger under equivalent conditions.

2. A method for increasing the amount of glucose in a syrup produced by saccharifying a starch substrate, comprising contacting the substrate with a glucoamylase having at least two, at least three, or at least four times more activity on an α-1,6 bond-containing substrate compared to the glucoamylase from Aspergillus niger under equivalent conditions, wherein the saccharifying with the glucoamylase produces a glucose syrup having a higher level of glucose compared to saccharifying the same starch substrate with the glucoamylase from Aspergillus niger under equivalent conditions.

3. The method of claim 1 or 2, wherein the α-1,6 bond-containing substrate is amylopectin, panose or isomaltose.

4. The method of any of the preceding claims, wherein the glucoamylase has at least 20% more activity at pH 4.5 on soluble starch substrate compared to the glucoamylase from Aspergillus niger under equivalent conditions.

5. The method of any of the preceding claims, wherein the glucose syrup comprises at least 4%, at least 10%, or at least 25% more glucose compared to a syrup produced by saccharifying with the glucoamylase from Aspergillus niger at a temperature between 60 and 69° C.

6. The method of any of the preceding claims, wherein the glucose syrup comprises at least a 4% reduction, at least a 10% reduction, or at least a 20% reduction in DP3+ compared to a glucose syrup prepared by contacting the same starch substrate with the glucoamylase from Aspergillus niger under equivalent conditions.

7. The method of any of the preceding claims, wherein the glucose syrup comprises at least 90% glucose, at least 91% glucose, at least 92% glucose, at least 93% glucose, at least 94% glucose, at least 95% glucose, at least 96% glucose, at least 97% glucose, at least 98% glucose or at least 99% glucose.

8. The method of any of the preceding claims, wherein saccharifying the starch substrate is performed at a temperature above 60° C., above 65° C., above 70° C., above 75° C., or above 80° C.

9. The method of any of the preceding claims, wherein saccharifying the starch substrate is performed at a pH below 4.5, below 4.0, or below 3.5.

10. The method of any of the preceding claims, performed within a simultaneous saccharification and fermentation process.

11. The method of any of the preceding claims, wherein the glucoamylase has at least 50% residual activity at 80° C. after 10 minutes at pH 5.0.

12. The method of any of the preceding claims, wherein the glucoamylase has at least 20% more activity at pH 3 compared to the glucoamylase from Aspergillus niger under equivalent conditions.

13. The method of any of the preceding claims, wherein the glucoamylase is from Penicillium glabrum, Symbiotaphrina kochii, Penicillium brasilianum or a variant, thereof.

14. The method of any of the preceding claims, wherein the glucoamylase is selected from the groups consisting of:
d) a polypeptide having the amino acid sequence of SEQ ID NO: 2, SEQ ID NO: 4, or SEQ ID NO: 6;
e) a polypeptide having at least 80% identity to the amino acid sequence of SEQ ID NO: 2, SEQ ID NO: 4, or SEQ ID NO: 6; or
f) a polypeptide having at least 80% identity to a catalytic domain of SEQ ID NO: 2, SEQ ID NO: 4, or SEQ ID NO: 6.

15. A recombinant construct comprising a nucleotide sequence encoding a glucoamylase, wherein said coding nucleotide sequence is operably linked to at least one regulatory sequence functional in a production host and is selected from the group consisting of the nucleotide sequence set forth in SEQ ID NO: 1, SEQ ID NO: 3, or SEQ ID NO: 5 or a nucleotide sequence with at least 80% sequence identity thereto, wherein said regulatory sequence is heterologous to the coding nucleotide sequence, or said regulatory sequence and coding sequence are not arranged as found together in nature.
